(19) United States
(12) Patent Application Publication
(10) Pub. No.: US 2020/0069170 A1
(43) Pub. Date: Mar. 5, 2020

(54) MATHIS LAPAROSCOPIC INGUINAL HERNIA ILLUMINATOR

(71) Applicant: Mathis Miller, Warsaw, IN (US)

(72) Inventor: Mathis Miller, Warsaw, IN (US)

(21) Appl. No.: 16/559,696

(22) Filed: Sep. 4, 2019

Related U.S. Application Data
(60) Provisional application No. 62/726,479, filed on Sep. 4, 2018.

(51) Int. Cl. A61B 1/313; A61B 1/07; A61B 1/06; A61B 1/05; A61B 1/015; A61M 13/00

(52) U.S. Cl. CPC A61B 1/3132; A61B 1/07; A61M 13/003; A61B 1/05; A61B 1/015; A61B 1/0684

(57) ABSTRACT

An apparatus for a surgery that has a sleeve extending between a first end and a second end and defining a passage there through, a first band extending from the first end of the sleeve and connecting the sleeve to a light source, and at least one fiber optic filament defined at least partially in the first band and being incorporated into the sleeve, the fiber optic filament extending from the light source. The fiber optic filament extends from the light source to the second end of the sleeve to thereby direct light generated by the light source out of the second end.

### CROSS-REFERENCE TO RELATED APPLICATIONS

The present disclosure claims the benefit of U.S. Provisional Application No. 62/726,479 filed on Sep. 4, 2018.

### TECHNICAL FIELD

The present teachings are related to the illumination and insufflation of an inguinal hernia sac into the preparitoneal space from an intra-abdominal approach through a laparoscopic port.

### BACKGROUND

The goal of a total extraperitoneal (“TEP”) laparoscopic inguinal hernia repair is to locate and identify the hernia sac, followed by the reduction of the sac, and the placement of mesh in the preparitoneal space. In the performance of a TEP laparoscopic inguinal hernia repair, a supraumbilical incision is made followed by the insertion of a laparoscopic port into the abdominal cavity. A camera lens is then inserted through the port and into the abdominal cavity where the inguinal hernia is located and confirmed. An infraumbilical incision in the anterior fascia is made lateral to the linea alba exposing the posterior rectus sheath. A balloon dissector or a laparoscopic atraumatic dissector is then used to open up the preperitoneal space under direct visualization from the camera. An angled scope can be used to place two additional ports under direct visualization in the infraumbilical midline. Blunt dissection is then used to expose and identify landmarks among surrounding tissue and eventually the hernia sac. Care is taken to avoid injury to the area containing critical vessels, cord structures, and nerves known in the industry as the “Triangle of Doom,” “Triangle of Pain,” and “Hesselbach's Triangle.” Once the hernia sac is located and identified, it is then reduced and a mesh is placed.

In the prior art, the process of dissecting through the preperitoneal space in search of the hernia sac is critical and time consuming due to the risk of injury to major surrounding structures. It is not unusual to be misled in identifying the hernia sac's location during the dissection due to common appearances of tissues and scarring.

The present disclosure allows for immediate identification of the hernia sac's location, whether direct, indirect, or femoral, upon entering the preparitoneal space among other things.

### SUMMARY

This disclosure relates to laparoscopic surgery and in particular to an apparatus and method employed in laparoscopic herniorrhaphy, wherein the laparoscopic tissue structure is identified via illumination by the apparatus. The laparoscopic inguinal hernia illuminator comprises a flexible fiber optic sleeve that is made of three interconnected components. The control end of the illuminator includes a battery powered, push-button LED light source that provides illumination to the device's fiber optic filaments. Stemming from the light source are two divided semi-rigid fiber optic bands. The bands carry the fiber optic filaments from the light source to the fiber optic sleeve, and provide stability for advancing the sleeve along a camera lens placed in an intra-abdominal laparoscopic port and into the hernia sac. The bands are also used to withdraw the sleeve from the intra-abdominal space and out of a laparoscopic port. The bands are divided in two parallel segments to provide a space for a camera head to be advanced to the sleeve's proximal opening, and still allow the sleeve to be advanced along the lens and through the intra-abdominal space. Continuing from the bands is a flexible fiber optic sleeve. The sleeve's function is to be advanced along a laparoscopic camera lens, through the intra-abdominal space, and into an inguinal hernia sac. Incorporated into the sleeve are the fiber optic filaments which transmit light through to the distal end of the sleeve providing illumination to the hernia sac. The sleeve itself is to be inserted into a 5 mm laparoscopic port while, in addition, the sleeve's channel allows for a 5 mm laparoscopic camera lens to be advanced through it. The sleeve may also be used as a channel by which direct insufflation to the hernia sac may be performed.

In operation, a 5 mm laparoscopic camera lens may be inserted into the illuminator's flexible fiber optic sleeve until the camera head contacts the proximal opening of the sleeve. The lens and sleeve may then be inserted through an intra-abdominal 5 mm laparoscopic port and into the insufflated abdominal cavity. The lens and sleeve may then be advanced through the abdominal cavity to the opening of the inguinal hernia where the hernia is then identified. The illuminator's semi-rigid bands are then grasped by hand and used to advance the sleeve further into the abdominal cavity, gliding beyond the distal tip of the camera lens, and gently presenting the distal portion of the flexible sleeve into the hernia sac. At any time leading up to this point, the illuminator's fiber optic light source can be activated. With the light source activated, the illuminator's sleeve is left in place with its distal end resting in the inguinal hernia sac. While holding fast the illuminator's semi-rigid fiber optic bands, the camera and lens are then drawn back out of the illuminator's sleeve, abdominal cavity, and laparoscopic port, leaving the distal end of the illuminator's sleeve in the hernia sac with its fiber optic filaments illuminating the hernia sac. Steps are then taken to enter the preperitoneal space where the hernia sac is located and the hernia sac may be immediately located and identified because it is illuminated by the illuminator. Blunt dissection may then be performed to advance toward the desired location, reduce the hernia sac, and provide space for the placement of a mesh patch.

With the illuminator's sleeve resting in the hernia sac, the sleeve can then be used as a channel between the intra-abdominal laparoscopic port and the hernia sac to directly insufflate the sac into the preparitoneal space from an intra-abdominal approach. This can be performed at any time during dissection of the preparitoneal space, reduction of the sac, and placement of mesh. Insufflation of the sac can aid in placing the mesh by identifying the hernia sac's reduced position. The aid of the illuminator will also reduce the risk of mistakenly dissecting through the peritoneal membrane.

Accordingly, it is an object of this disclosure to provide a new apparatus for identifying the location and position of hernia sac tissue in laparoscopic surgery.

Another object of this disclosure is to provide a way to illuminate an inguinal hernia sac in the preparitoneal space from an intra-abdominal approach with the use of the flexible fiber optic sleeve so that the tissue may be located and identified safer, faster, and more efficiently throughout an operation. This greatly reduces the risk of injury to critical structures due to the elimination of needless and or mistaken dissection.

A further object of this disclosure is to provide a new way to directly insufflate a hernia sac into the preparitoneal space from an intra-abdominal approach by using the sleeve as a channel for insufflation, while maintaining a direct view into the insufflated preparitoneal space. This will aid in identifying the hernia sac's reduced position, which in turn aids in the positioning of the implanted mesh.

In one non-exclusive example from the present disclosure, the immediate identification of the hernia sac's location upon entering the preparitoneal space is accomplished with the illumination of the hernia sac from an intra-abdominal approach, which in turn provides a safer, faster, and more efficient dissection of the surrounding tissues as well as the reduction of the sac itself. Further approximation of the hernia sac's positioning can be observed by using devices and methods disclosed herein to create a channel through which direct insufflation of the sac may be performed in the preparitoneal space from the intra-abdominal approach. The illumination and insufflation of the sac can be performed before, during, and with the placement of a mesh.

### DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 is a front view of one embodiment of an illuminator of this disclosure with portions cutaway;

FIG. 2a is an illustrative view of an intra-abdominal area with three possible herniated areas of an inguinal canal in which the illuminator of FIG. 1 can be inserted;

FIG. 2b is an illustrative view of the illuminator of FIG. 1 entering an indirect inguinal hernia;

FIG. 2c is an illustrative view of the illuminator of FIG. 1 entering a direct inguinal hernia;

FIG. 2d is an illustrative view of the illuminator of FIG. 1 entering a femoral hernia;

FIG. 3a is an illustrative view of a laparoscopic camera inserted into a sleeve of the illuminator of FIG. 1, both being inserted simultaneously through an intra-abdominally placed port, and presenting the illuminator's sleeve into the opening of the inguinal hernia;

FIG. 3b is an illustrative view of a camera lens being withdrawn from the sleeve of the illuminator of FIG. 1 and leaving the distal portion of the sleeve at rest in the hernia sac with the illuminator's light activated;

FIG. 3c is an illustrative view of the distal portion of an illuminator's sleeve at rest in a hernia sac with the illuminator's light activated while two other working ports are directed toward the hernia sac from in the preparitoneal space;

FIG. 3d is an illustrative view of the illuminator of FIG. 1 working as a channel to directly insufflate the hernia sac into the preparitoneal space from an intra-abdominal approach;

FIG. 4a is an illustrative view of a laparoscopic camera lens entering the preparitoneal space with the distal portion of an illuminator's sleeve illuminating a hernia sac from an intra-abdominal approach;

FIG. 4b is an illustrative view of the illuminator of FIG. 1 in a hernia sac in relation to major vessels, cord structures, triangle of doom, triangle of pain, and hesselbach triangle;

FIG. 5a is a cross sectional view of a distal end of a sleeve of the illuminator of FIG. 1; and

FIG. 5b is a cross sectional view along the line 5-5 of FIG. 1 at the point the bands join the sleeve at the sleeve's proximal end.

Corresponding reference numerals are used to indicate corresponding parts throughout the several views.

### DETAILED DESCRIPTION

The above-mentioned aspects of the present application and the manner of obtaining them will become more apparent and the teachings of the present application itself will be better understood by reference to the following description of the embodiments of the present application taken in conjunction with the accompanying drawings.

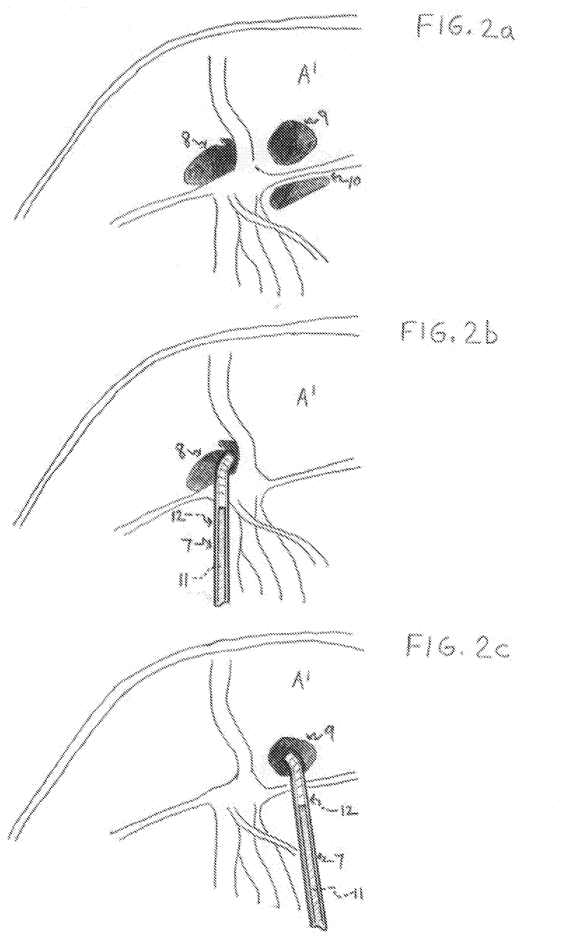

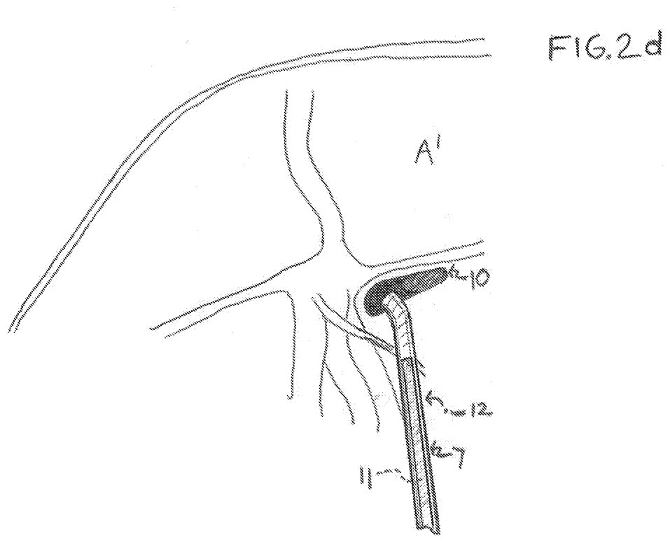

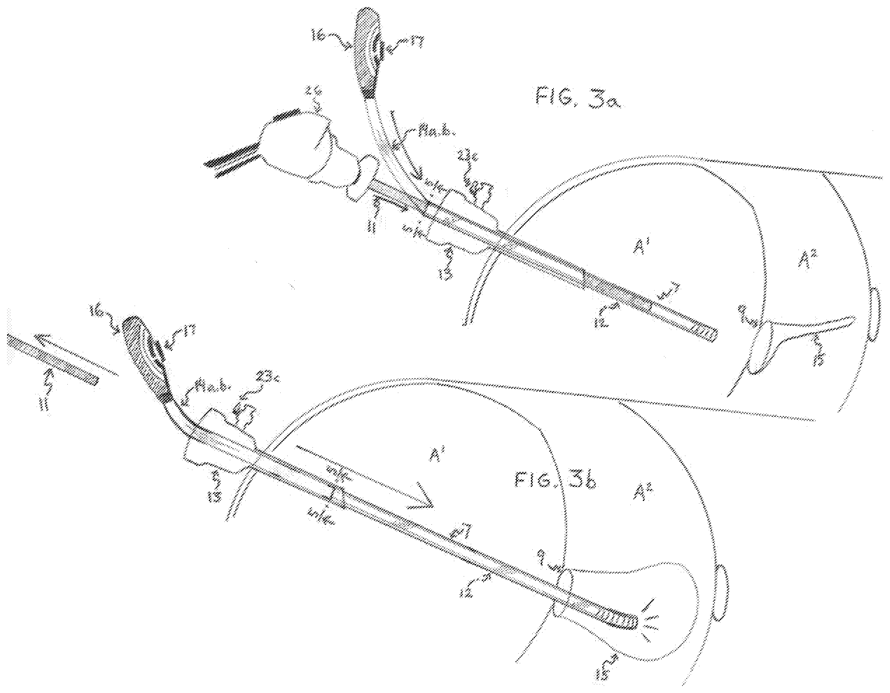

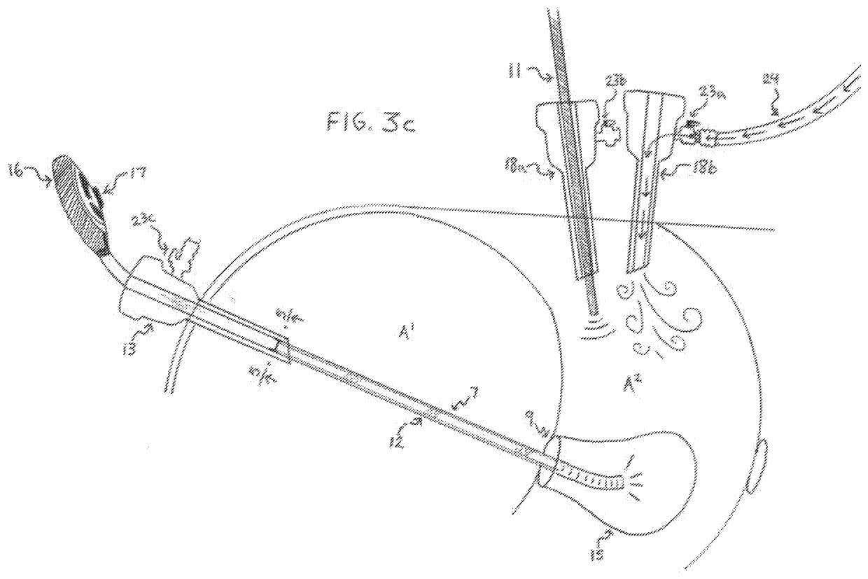

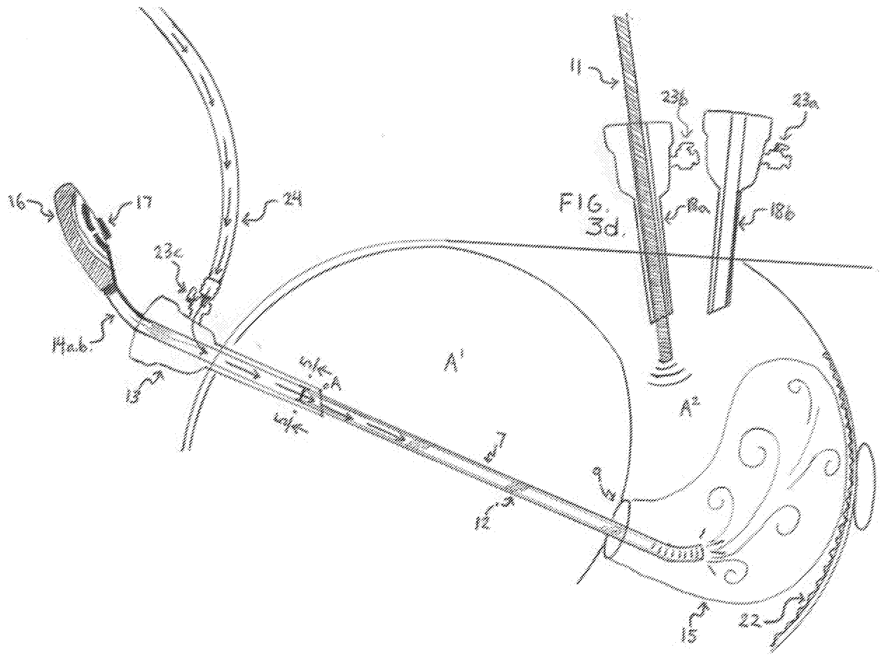

Referring now to the drawings, the Mathis Laparoscopic Inguinal Hernia Illuminator 7 is used particularly in laparoscopic herniorrhaphies, wherein the illuminator 7 allows for immediate identification of the hernia sac's 15 location, whether indirect 8, direct 9, or femoral 10, upon entering the preparitoneal space A2 as shown in FIGS. 2a-d. This is accomplished with the illumination of the hernia sac 15 by the illuminator 7, which in turn provides a safer, faster, and more efficient dissection of the surrounding tissues as well as the reduction of the sac 15 itself, see FIG. 3c. Further approximation of the hernia sac's 15 positioning can be observed by using the illuminator 7 as a channel through which direct insufflation of the sac 15 may be performed in the preparitoneal space A2 from the intra-abdominal A1 approach, see FIG. 3d. The illumination and insufflation of the sac 15 can be performed before, during, and with the placement of a mesh 22 as shown in FIG. 3d.

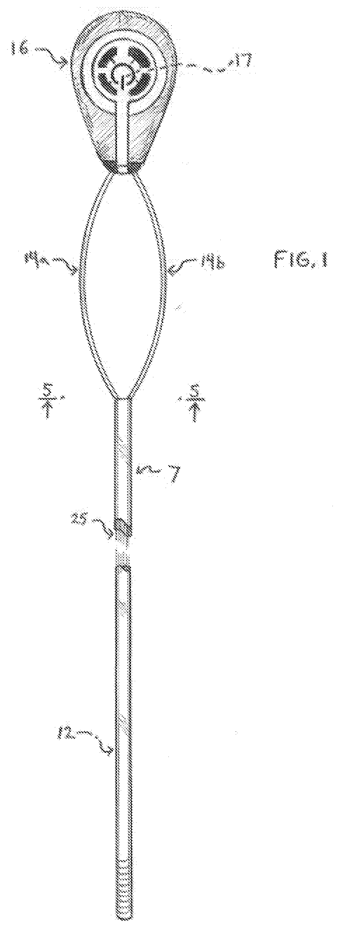

The illuminator 7 is shown in FIG. 1 and includes a flexible fiber optic sleeve 12 that may be made of three interconnected components. The control end of the illuminator 7 includes a battery powered, push-button 17 LED light source 16 that provides illumination to the device's fiber optic filaments 25. Stemming from the light source 16 are two divided semi-rigid fiber optic bands 14a and 14b. The bands 14a and 14b, carry the fiber optic filaments 25 from the light source 16 to the fiber optic sleeve 12, and provide stability for advancing the sleeve 12 along a camera lens 11 placed in an intra-abdominal laparoscopic port 13 and into the hernia sac 15.

The bands 14a and 14b, are also used to withdraw the sleeve 12 from the intra-abdominal space A1 and out of a laparoscopic port 13. The bands 14a and 14b are divided in two parallel segments to provide a space for a camera head 26 to be advanced to the sleeve's proximal opening 5-5, and still allow the sleeve 12 to be advanced along the lens 11 and through the intra-abdominal space A1. Continuing from the bands 14a and 14b may be the flexible fiber optic sleeve 12. The sleeve 12 may be advanced along a laparoscopic camera lens 11, through the intra-abdominal space A1, and into an inguinal hernia sac 15. Incorporated into the sleeve 12 are fiber optic filaments 25 which transmit light through to the distal end of the sleeve 12 providing illumination to the hernia sac 15. The sleeve 12 itself is to be inserted into a laparoscopic port while, in addition, the sleeve's channel 27 allows for a laparoscopic camera lens 11 to be advanced there through. The sleeve 12 may also be used as a channel by which direct insufflation to the hernia sac 15 may be performed. In other words, the sleeve 12 may provide a substantially fluidly sealed passage that extends from the bands 14a, 14b to a distal end of the sleeve 12.

In one aspect of this disclosure, the bands 14a, 14b may have a length that is less than the length of the laparoscopic port 13 to ensure that the sleeve 12 does not become positioned entirely outside of the distal opening of the laparoscopic port 13. In this configuration, at least a portion of the sleeve 12 remains within the laparoscopic port 13 even when the illuminator 7 is inserted fully into the laparoscopic port 13. More specifically, a portion of the housing of the light source 16 may contact a proximal end of the laparoscopic port 13 before the sleeve 12 exits the distal end of the laparoscopic port 13. With the bands 14a, 14b sized to remain within the laparoscopic port 13, the risk of damaging tissue when withdrawing the illuminator 7 from the laparoscopic port 13 is reduced. In one non-exclusive example, the bands 14a, 14b have a length that is about the same, or less than the length of the laparoscopic port 13. In another non-exclusive example, the length of the bands 14a, 14b may be about nine to thirteen centimeters. In yet another example, the length of the bands 14a, 14b is about eleven centimeters.

Similarly, the length of the sleeve 12 may be such that allows the sleeve 12 to extend from the distal end of the laparoscopic port 13 at least partially into the hernia sac 15 as illustrated in FIG. 3b. While the sleeve 12 may be different lengths to accommodate different anatomies, in one non-exclusive example the sleeve 12 is between about twenty-three and thirty-three centimeters long. In yet another example, the sleeve 12 is about twenty-eight centimeters long. A person skilled in the art understands that the length of the bands 14a, 14b and the sleeve 12 are such to allow the distal end of the sleeve 12 to at least partially enter the hernia sac 15 without requiring the bands 14a, 14b to exit the distal end of the laparoscopic port 13. While specific dimensions are given herein as examples, this disclosure considers applying these teachings to different portions of the anatomy wherein different dimensions of the sleeve 12 and bands 14a, 14b would be required.

Further, the sleeve 12 and bands 14a, 14b may have a thickness that allows the sleeve 12 and bands 14a, 14b to be positioned in the annular cavity between the laparoscopic port 13 and the camera lens 11. More specifically, the camera lens 11 may be substantially cylindrical in shape and have about a five millimeter diameter. Similarly, the laparoscopic port 13 may define a cylindrical cavity there through that has about a seven millimeter diameter. In this particular configurations, the sleeve 12 may be sized to occupy the annular cavity between the camera lens 11 and the laparoscopic port 13. Similarly, the bands 14a, 14b may have a thickness that is less than the thickness of the annular cavity to thereby allow the bands 14a, 14b to be positioned between the camera lens 11 and the laparoscopic port 13 as well.

FIGS. 2a-d illustrate the possible locations of which a hernia may located and identified intra-abdominally A1 as either an indirect 8, direct 9, and or a femoral hernia 10. FIG. 3a depicts a laparoscopic camera lens 11 inserted into the flexible fiber optic sleeve 12 of the illuminator 7, both are advanced into an intra-abdominal port 13, through an insufflated abdominal cavity A1, and presented to the opening of the hernia 9. The illuminator's 7 semi-rigid fiber optic bands 14a and 14b are then grasped together between the operator's index finger and thumb and are advanced down into the port 13, along the camera lens 11, through the hernia's opening, and into the hernia sac 15, see FIG. 3b. Holding the illuminator 7 by the light source 16, the power button 17 is pressed, activating the LED bulb and illuminating the fiber optic filaments 25. With the light source 16 activated, the distal end of the fiber optic sleeve 12 illuminates the hernia sac 15.

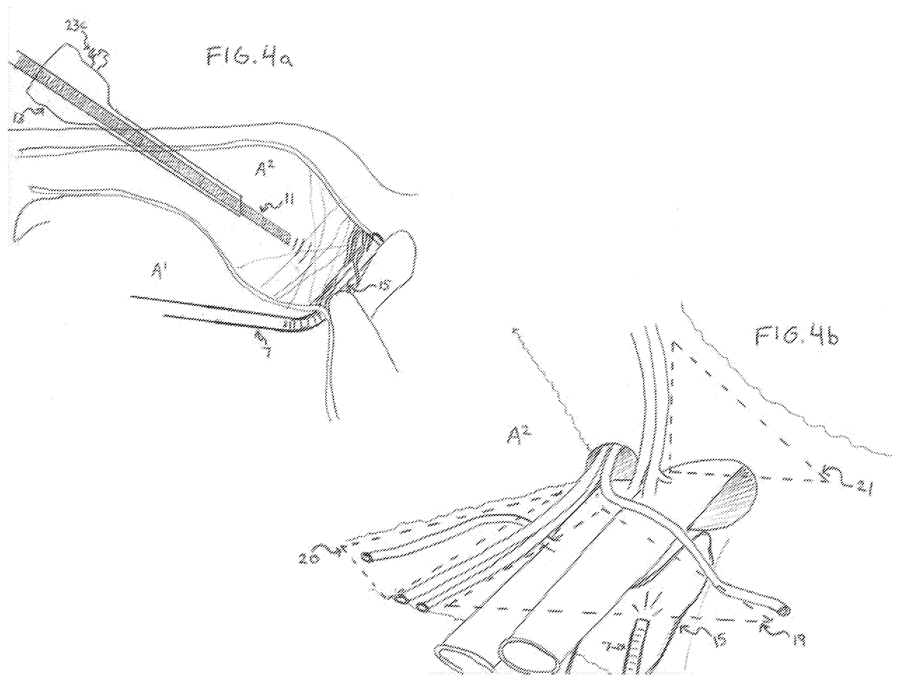

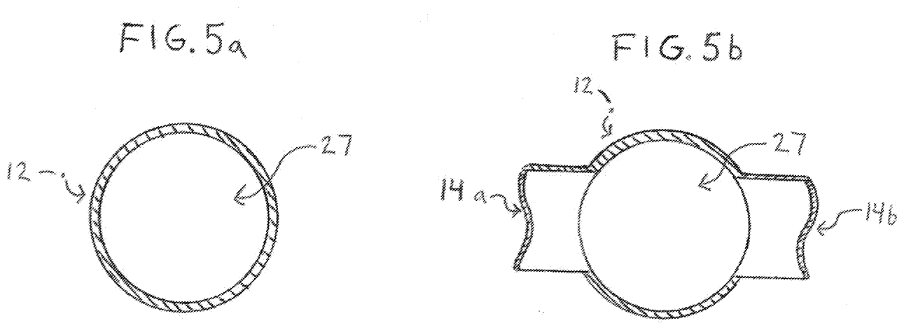

The light source 16 remains to be held fast while the camera lens 11 is then drawn back out of the sleeve 12 and the port 13, and free of the abdomen as shown in FIG. 3b. The preparitoneal space A2 is now entered and insufflated using additional laparoscopic ports 18a and 18b. With additional ports allowing access into the preparitoneal space A2, and the illuminator's lights source 16 activated, the location and position of the hernia sac 15 in the preparitoneal space A2 is immediately identified (see FIG. 3c). Blunt dissection of the preparitoneal space A2 is then used to expose and identify landmarks among surrounding tissue and eventually the hernia sac 15. Care is taken to avoid injury in the “Triangle of Doom” 19, Triangle of Pain” 20, and “Hesselbach's Triangle” 21 which collectively contain critical vessels, cord structures, and nerves (see FIGS. 4a-4b). Once the hernia sac 15 is located and identified, it is then reduced and a mesh 22 as shown in FIG. 3d is placed in the preparitoneal space A2.

Further approximation of the hernia sac's 15 positioning can be observed by using the illuminator 7 as a channel through which direct insufflation of the sac 15 may be performed in the preparitoneal space A2 from the intra-abdominal A1 approach shown in FIG. 3d. Insufflation of the hernia sac 15 is performed by first insufflating the preparitoneal space A2, then closing the accessing port's insufflation valves 23a and 23b followed by disconnecting the CO2 insufflation tubing 24, see FIG. 3c. Then the CO2 tubing 24 is attached to the intra-abdominal port's insufflation valve 23c while the illuminator's sleeve 12 remains in the hernia sac 15, see FIG. 3d. With the illuminator sleeve's proximal end 5-5 advanced in the port 13 distal to the port's insufflation valve 23c, and proximal to the port's distal tip at point A FIG. 3d, this will provide a channel that will carry CO2 gas directly to the hernia sac 15, bypassing the intra-abdominal cavity A1 as shown in FIG. 3d. With the laparoscopic camera lens 11 inserted into the insufflated preparitoneal space A2, the hernia sac 15 can be observed inflating and deflating by opening and closing the intra-abdominal port's insufflation valve 23c, see FIG. 3d. The illumination and insufflation of the sac 15 can be performed before, during, and with the placement of a mesh 22, see FIG. 3d.

The added risks and consumption of time with searching for the hernia sac 15 are thereby eliminated. Presently, the process of dissecting through the preperitoneal space A2 in search of the hernia sac 15 is critical and time consuming due to the risk of injury to major surrounding structures. It is not unusual to be misled in identifying the hernia sac's 15 location during the dissection due to common appearances of tissues and scarring. Complications that may evolve with an improperly positioned mesh 22 are also reduced due to the ability of the illuminator 7 to directly insufflate the hernia sac 15, revealing the hernia sac's presentation.

Upon completion of the repair, or any point before, the illuminator 7 may be removed by grasping the semi-rigid bands 14a and 14b and gently withdrawing it back out of the laparoscopic port 13 and free of the abdomen. The position of the sleeve 12 within the hernia sac 15 may also be finely adjusted under direct visualization by reinserting the camera lens 11 through the port 13 and into the sleeve 12. By insufflating the abdominal cavity A1 the sleeve can then be manipulated by controlling the semi-rigid bands 14a and 14b.

While two semi-rigid bands 14a, 14b have been described herein throughout, this disclosure considers any number of rigid bands for providing illumination to the distal end of the sleeve 12. More specifically, a single semi-rigid band may extend from the light source 16 to the tube and house one or more fiber optic cables therein. Further still, in yet another embodiment there may be more than two semi-rigid bands extending between the light source 16 and the sleeve 12. Accordingly, this disclosure considers using any number of semi-rigid bands.

While the illuminator 7 is described throughout with specific reference to laparoscopic herniorrhaphies, this disclosure contemplates other uses for the illuminator 7 as well. Accordingly, the teachings described herein are intended to be applied to any type of surgical procedure or the like that may benefit from these teachings. Accordingly, the specific surgical procedure and methods described herein are meant only to be exemplary, and other applications not specifically discussed herein are also considered.

Further, in one aspect of this disclosure the illuminator 7 and other devices and methods discussed herein may be utilized for a robotic surgery wherein the illuminator 7 is utilized as part of a robotic system. Alternative, medical staff such as doctors, nurses, and the like may implement the teachings discussed herein.

While this disclosure has been described with respect to at least one embodiment, the present disclosure can be further modified within the spirit and scope of this disclosure. This application is therefore intended to cover any variations, uses, or adaptations of the disclosure using its general principles. Further, this application is intended to cover such departures from the present dis-closure as come within known or customary practice in the art to which this disclosure pertains.

What is claimed is:

1. An apparatus for a surgery comprising:
a sleeve extending between a first end and a second end and defining a passage there through;
a first band extending from the first end of the sleeve and connecting the sleeve to a light source; and
at least one fiber optic filament defined at least partially in the first band and being incorporated into the sleeve, the fiber optic filament extending from the light source;
wherein, the fiber optic filament extends from the light source to the second end of the sleeve to thereby direct light generated by the light source out of the second end.

2. The apparatus of claim 1, further wherein the light source is a battery powered LED directed to emit light towards one end of the at least one fiber optic filament.

3. The apparatus of claim 1, further comprising a second band extending from the first end of the sleeve and connecting the sleeve to the light source, wherein the first and second band are semi-rigid.

4. The apparatus of claim 3, further wherein the first and second band each carry at least one fiber optic filament from the light source to the sleeve and provide a structure by which the sleeve may be manipulated.

5. The apparatus of claim 4, further wherein, the first and second bands are transparent and the illuminated fiber optic filaments are visible through the first and second bands.

6. The apparatus of claim 1, further wherein the sleeve is formed of a flexible material and configured to contour with the path of a hernia canal.

7. The apparatus of claim 1, further wherein the sleeve is transparent and the at least one fiber optic filament is visible through the sleeve.

8. The apparatus of claim 7, further wherein the transparent sleeve allows at least some peripheral visualization from a camera lens.

9. A kit for a laparoscopic surgery, comprising;
a sleeve having a passage defined therein and extending between a first end and a second end;
a camera lens sized to pass through the passage;
a fiber optic filament extending between the first and second end and defined at least partially in a wall of the sleeve;
a light source positioned to illuminate a proximal end of the filament to project light from a distal end of the filament;
wherein, when the light source illuminates the proximal end of the filament the filament projects light from the second end of the sleeve.

10. The kit of claim 9, further comprising a port defining a port passage there through, wherein the sleeve has an outer dimension that is sized to allow the sleeve to pass through the port passage.

11. The kit of claim 10, further wherein the sleeve and camera lens are sized to pass through the port passage when the camera lens is positioned within the passage of the sleeve.

12. The kit of claim 10, further wherein the passage between the first end and the second end is a substantially fluid-tight passage.

13. The kit of claim 12, further comprising:
a plug positioned between the light source and the distal end of the filament and sized to fit in a proximal end of the port passage; and
a valve defined in the port to selectively provide fluid to the port passage;
wherein, when the plug is positioned in the proximal end of the port passage fluid introduced through the valve travels through the passage of the sleeve and exits the second end of the sleeve.

14. The kit of claim 13, further wherein the light source is coupled to the sleeve with a band, wherein the filament travels from the light source to the sleeve through the band.

15. The kit of claim 14, further wherein the band has a thickness that allows the band to be positioned in the port passage without obstructing passage of the camera lens there through.

16. A method of illuminating an inguinal hernia sac in laparoscopic surgery, comprising:
providing a light source that directs light out an end of a sleeve, a laparoscopic camera lens sized to pass through the sleeve, and a port sized to allow the sleeve and camera lens to pass there through;
inserting the sleeve and camera lens through the port;
operating the light source to illuminate the end of the sleeve; and
advancing the sleeve through the port to present the end of the sleeve into a hernia sac to illuminate the hernia sac.

17. The method of claim 16, further comprising directly insufflating the hernia sac by removing the camera lens from the sleeve, plugging a proximal end of the port, and introducing fluid into the hernia sac through the sleeve via a valve in the port.

18. The method of claim 16 further comprising visualizing the illuminated hernia sac in a preparitoneal space.

19. The method of claim 16, further wherein the light source directs light out the end of the sleeve with one or more fiber optic filament defined partially through the sleeve.

20. The method of claim 18, further wherein the fiber optic filament passes from the light source through a band before entering the sleeve.
